(19) United States
(12) Patent Application Publication
(10) Pub. No.: US 2012/0157920 A1
(43) Pub. Date: Jun. 21, 2012

(54) Distributed processor configuration for use in infusion pumps

(75) Inventors: Eric J. Flachbart, Newport Center, VT (US); Todd Leadbeater, Saint Johnsbury, VT (US); John Wojdak, Brookfleld, MA (US); Rolf Eric Zuk, Monroe, NH (US); Duane E. Allen, Sheffield, VT (US)

(73) Assignee: Numia Medical Technology, LLC, Lyndonville, VT (US)

(21) Appl. No.: 13/327,927

(22) Filed: Dec. 16, 2011

Related U.S. Application Data
(60) Provisional application No. 61/423,794, filed on Dec. 16, 2010.

(51) Int. Cl. A61M 5/168

(52) U.S. Cl. 604/151

(57) ABSTRACT

The present invention provides an infusion pump control system comprises a plurality of computing components positioned on discrete hardware modules to complete an infusion task. Those discrete processors, which are internally redundant and communicate through a common medium that provides for redundancy of the communication ability to react to internal failures in a known manner, implement capabilities specific to infusion pump functions, to complete an infusion task. Also, this invention provides automatically switchable redundant power supplies and a new mechanism for firmware provisioning using multi-dropped JTAG for a plurality of computing components.

### CROSS-REFERENCE TO A RELATED APPLICATION

The present application claims priority to provisional U.S. patent application Ser. No. 61/423794, filed on Dec. 16, 2010, which is assigned to the assignee of the present application and incorporated herein by reference.

### BACKGROUND OF THE INVENTION

This invention is related to a distributed processor configuration to provide an easily-maintained, scalable, critically redundant, intelligent operation system for use in infusion pumps.

The current architectures of medical infusion pumps utilize either a single processor or multiple processors adopting a master/slave communication mechanism to operate an infusion pump that does not allow nodes to equally access to a communication bus. Such a design is disadvantaged by several distinct issues that compromise operational integrity and ease of ongoing technological maintenance & development. These issues include specialization of processing module function to the exclusion of any form of redundant processing and monolithic code-bases (firmware) that are harder to maintain. A lack of any real hardware-based redundancy, which is truly effective in the face of faults/failures during life-critical operation, and a lack of software-based fault handling that takes advantage of hardware redundancy make it impossible to complete entire mission-critical functions prior to a complete device failure. The problems associated with monolithic code-bases are an inability to provide a scalable environment that promotes product expansion or reusable design/technology to easily create product variants, difficult maintenance and a high level of difficulty for a single engineer to completely conceptualize the device without extraordinary supports. The present invention provides an architectural methodology that directly addresses the above issues.

### SUMMARY

An infusion pump control system having at least one syringe, motor, at least one user-interface components, and at least one infusion pump-interface components, comprises a plurality of computing components discretely modularized to implement capabilities specific to infusion pump functions, and at least one communication bus, wherein the plurality of computing components communicate each other through the at least one communication bus.

In another embodiment, the infusion pump control system also includes at least one sensor bus, to which the at least one user-interface components and at least one infusion pump-interface components are connected, where the plurality of computing components collects data from the at least one user-interface components and at least one infusion pump-interface components.

In another embodiment, at least one of the plurality of computing component is redundant and the redundant counterpart is able to take the role of at least one of the plurality of computing component to complete an infusion when the at least one of the plurality of computing component fails.

In another embodiment, the infusion pump control system comprise a power bus capable of automatically selecting remained functional power supplie(s) from redundant power supplies when any of redundant power supplies fails.

In another embodiment, the infusion pump system comprise a system management bus providing for a multi-dropped JTAG coupling with a multi-dropped JTAG selection mechanism capable of providing firmware provisioning for the plurality of computing components.

These and other features, aspects, and advantages of the present invention will become better understood with reference to the following description, appended claims, and accompanying drawings.

### BRIEF DESCRIPTION OF THE DRAWINGS

FIG. 1 shows major components of an infusion pump control system.

FIG. 2 is a bus layout.

FIG. 3 shows a redundancy implementation of the user-interface processor, the application processor, the delivery processor, and the safety processor.

FIG. 4A illustrates a scenario when both the application processor and the safety processor fails, the user-interface processor take over an additional role of the application processor and the delivery processor takes over an additional role the safety processor.

FIG. 4B illustrates a scenario when the delivery processor fails, the safety processor replace the delivery processor.

FIG. 4C illustrates a scenario when the delivery processor fails, the application processor replace the delivery processor while the safety processor take over the role of the application processor.

FIG. 4D illustrates a scenario when both the application processor and the delivery processor fail, the safety processor take over the role of the application processor and the delivery processor, or the safety processor takes on the role of the delivery processor while the user-interface processor takes in the role of the application processor.

FIG. 4E illustrates a scenario when the application processor fails, the safety processor takes over the role of the application processor,

FIG. 5A shows a redundant power system.

FIG. 6A demonstrates a multi-dropped JTAG selection mechanism.

FIG. 6B illustrates how a multi-dropped JTAG selection mechanism selects JTAG connection between local JTAG and system JTAG.

FIG. 6C is a layout of the system management bus.

FIG. 7 illustrates layout of a processor.

FIG. 8 illustrates a device level communication protocol of the communication bus.

FIG. 9 illustrates an application level communication protocol of the communication bus.

FIG. 10 is a flowchart showing a minimal scenario for firmware provisioning to utilize the content of local data store on the database processor.

### DETAILED DESCRIPTION OF EXEMPLARY EMBODIMENTS

The present invention provides an infusion pump control system, having pump interface components, user-interface components, a driving component, and at least one syringe, comprises a plurality of computing components positioned on discrete hardware modules to complete an infusion task. In an embodiment, the plurality of computing components are processors positioned on discrete hardware modules to complete the infusion task. Those discrete processors, which are internally redundant and communicate through a common medium that provides for redundancy of communications and the ability to react to internal failures in a known manner, implement capabilities specific to infusion pump functions, to complete an infusion task. Encapsulation of functions within discrete modules, combined with optional firmware provisioning, provides the advantages of scalability, reconfiguration/reuse of computing components to create product variants, an easy replacement/update, and easy maintenance by a single engineer without understanding the entire system. Support for hardware & software redundancy at both the module and system level that provides multiple levels of recovery under operational conditions. In essence, this design implements the concept of high availability processing for life-critical operations and provides a self-healing capability that handles single point failures without compromise and multiple failures up to the point of complete module isolation. However, the present invention does not require that all the advantageous features need be incorporated into every embodiment.

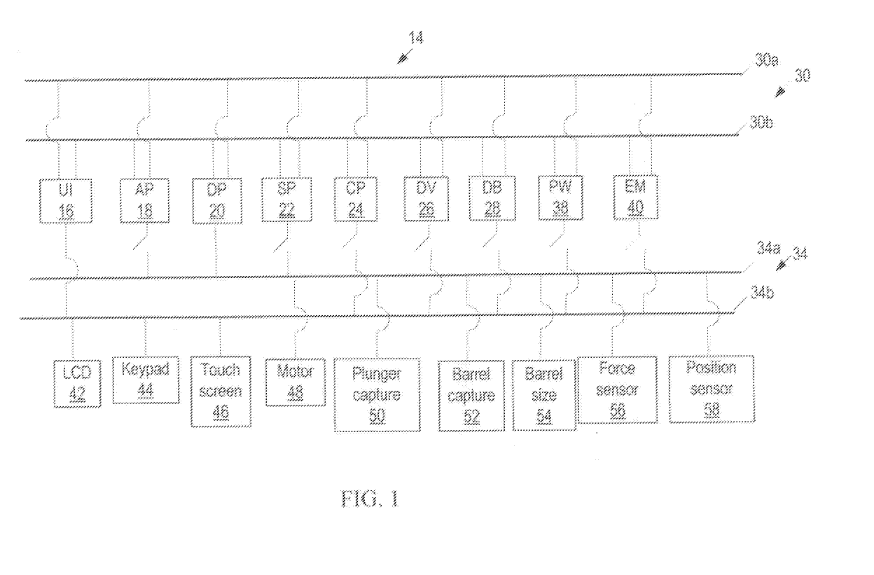

The major components of an infusion pump control system 14 are illustrated in FIG. 1.

In an embodiment, the infusion pump control system 14 further comprises a communication bus 30, and a sensor bus 34, and the plurality of processors are a user-interface device 16 (UI), a delivery processor 20 (DP), an application processor 18 (AP), and a safety processor 22 (SP).

The communication bus 30 provides a shared mastery that is able to be connected to each of the plurality of discrete processors and has a pair of redundantly identical data paths 30a and 30b.

However, the availability of the communication bus 30 to a processor of the plurality of processors is determined by the arbitration mechanism encapsulated in a separate patent application, the U.S. application Ser. No. 13327058 and filed on Dec. 15, 2011, which is incorporated herein by reference.

In the arbitration mechanism, an apparatus for controlling access of a plurality of the processors to the shared resource—the communication bus 30 comprises a comparator, an arbitrary reference signal preset in the comparator, and an aggregation component. Each of the plurality of processors that need access to the shared source is capable of adding a request signal to the aggregation component. The aggregation component adds up the request signals received from those processors of the plurality of processors that request access to the shared resource and generates a biased signal. The comparator compares the reference signal and the biased signal and generates a logic output signal either permitting an access to the shared resource or denying such access. Accordingly, a node that requests to access to the shared resource either is permitted to access to the shared resource or denied.

By using the arbitration mechanism, the communication bus 30 overcomes problems imposed by currently available multi-master bus systems that do not allow a plurality of nodes to equally access to the communication bus 30, although the present invention does nor require that foresaid advantageous feature be incorporated into every embodiment. The communication bus 30 supports both a node-to-node message passing, where a message is sent to a specific node, and a broadcast message passing, where a message is sent to all nodes.

The plurality of discrete computing components connect to and use the communication bus 30 to form a cooperating computational network. They can access the communication bus 30 only when it is available based on the arbitration mechanism.

The communication bus 30's two redundant data lanes 30a and 30b provide support for a fixed number of processors in a position independent of configuration. The communication bus 30 is designed such that under normal conditions, only the data lane 30a is in operation and under the condition that the data lane 30a malfunctions, the data lane 30b automatically starts operation.

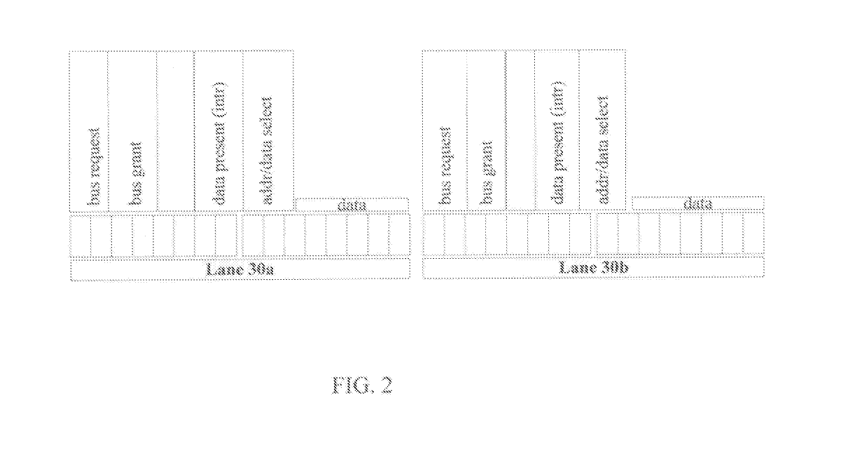

The layout of those date lanes 30a and 30b are shown in FIG. 2. They have a lane-change signal (out-bound) and a lane-select signal (in-bound). The data lanes 30a and 30b contain the multi-node arbitration signals, bus-request (out-bound) & bus grant (in-bound) and the data (send/recv) bits (addr/data selection bit and 8 data bits), any other signals as assigned by a designer.

As shown in FIG. 1, there is a singular set of signal paths by the sensor bus 34 for each of infusion pump-interface components including a motor 48, plunger capture 50, barrel capture 52, barrel size 54, force sensor 56, and position sensor 58, communicate with delivery processor 20 while user-interface components including LCD 42, a keypad 44, and touch 46, communicates with the user-interface processor 16. In other words, each sensor communicates with only one processor at one time by the sensor bus 34; only when such a processor connected to the sensor bus 34 fails, the another processor that is capable of replacing the functions of the failed processor is allowed to be connected to the sensor bus 34 by the arbitration mechanism. For example, when the delivery processor 20 is faulted, the application processor 18 connects to the sensor bus 34 by the arbitration mechanism.

Although in FIG. 1, only the motor 48, plunger capture 50, barrel capture 52, barrel size 54, force sensor 56, and position sensor 58 are shown to communicate with a sensor section 34a of the sensor bus 34, and only LCD 42, a keypad 44, and a touch screen 46 are shown to communicate with a section 34b of the sensor bus 34, both pump interface components and user-interface components communicating with the sensor bus 34 are not limited to those components aforementioned.

In an embodiment, in contrasted to the communication bus 30, the sensor bus 34 has only one sensor lane, the failure of an infusion pump-interface component is to be compensated by the redundant of such failed infusion pump-interface components in the infusion pump control system 14.

The user-interface processor 16 provides all display and user input supports for the pump.

The delivery processor 20 controls operation of all motors and sensors substantially essential to the driving/controlling/monitoring the infusion of the infusion pump control system 14. The delivery processor 20 communicates with the sensor bus 34 and it only disconnects with the sensor bus 34 when it malfunctions. The delivery processor 20 performs several tasks: generate a flow rate or bolus, a fixed flow-rate for a finite period of time or a fixed volume (bolus) in a specific period of time, and acquires, over the sensor bus 34, data central to the driving/controlling/monitoring the infusion of the infusion pump control system 14 such as sensor data, motor counts, motor state (on/off), and volume delivered, from pump interface components such as sensors of the motor 48, plunger capture 50, barrel capture 52, barrel size 54, and force sensor 56, and position sensor 58, and communicates such date to the other processors over the communication bus 30.

In an embodiment, the delivery processors 20 detects infusion-specific faults/alarms and reports such to the application 18 & safety processors 22 for further disposition through the communication bus 30.

The application processor 18 controls all functions involved in the operation of an infusion pump. From the time the pump is powered up until the pump is powered down, the application processor 18 controls the actions associated with pump usage. while the ancillary functions needed to affect the pump's application are provided by support processors in the configuration under the direction of the application processor 18, the application processor 18 acquires sensor data from delivery processor 20, user-generated events from user-interface processor 16, and communications-related information from the support processors to determine changes in the pump's execution state.

The primary tasks performed by the application processor are:
Startup: Initialization of the pump's resources (motor, sensors, communications, etc)
   Diagnosis to determine the pump's ability to properly perform
   Resumption of previous infusion tasks (if applicable)
Setup: Determination of the infusion parameters
   Preparation of the pump to perform an infusion
Operation: Management of the startup, progress, completion and restart of an infusion
   Direction (control) of the support processors that provide for the various aspects of the infusion (i.e. motor control, user-interface, sensor monitoring, communications, etc)
Control: Monitor internal (pump) and external (syringe, Iv set, clinician) events that affect an infusion and react to those events by directing the functions of the support processors
Safety: Determine alarm/alert states for the pump while operational (not limited to infusions)
Utility: Management of the various data sets involved in the execution of an infusion (drug library, syringe library, firmware load) and the historical/real-time information accumulated during an infusion while providing a means for the clinician/bio-med technician to adjust a defined set of operational parameters that affect pump operation
Shutdown: Accumulation of infusion parameters (run-time, historical) to support startup
   Close out of pump's resources to insure a safe state of conclusion

In an embodiment, the application processor 18 contains a finite state machine (FSM) that defines the pump's application, specifically, the FSM in the application processor 18 defines the state of the pump operation at any point in time. The FSM determines the transitions between pump states based on events it gathers from the other processors in the configuration and directs the functions of those processors to move the pump's activity forward.

The safety processor 22 monitors the application processor 18 and through communication bus 30, independently acquires data, user-generated events and communications-related information from processors other than the application processor 18 and the user-interface processor 16 to calculate pump state and infusion progress. The safety processor 22 compares aforesaid actual values with calculated values from the application processor 18 to determine if pump functions are proceeding in a correct and proper manner.

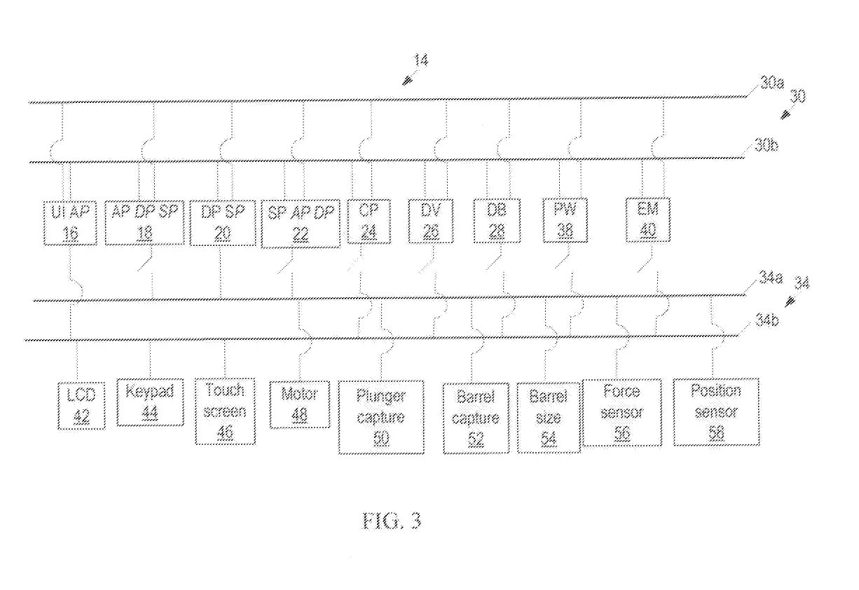

In order to complete an infusion under a situation where one or more processors malfunction, the proposed design applies distributed processor architecture in a manner that provides for at least sufficient redundancy within the infusion pump control system. Referring to FIG. 3, the application processor 18 has at least sufficient circuit and software of the delivery processor 20 and/or the safety processor 22 to takes over the task of the delivery processor 20 and the safety processor 22 when the delivery processor 20, the safety processor 22, or both malfunction. To complete an active infusion task, the delivery processor 20 that has at least sufficient circuit and software of the safety processor 22 takes on the additional role of the safety processor 22, when the safety processor 22 fails, while the user-interface processor 16 that has at least sufficient circuit and software of the application processor 18 takes on the additional role of the application processor 18 when the application processor 18 fails. Additionally, the safety processor 22 has at least sufficient circuit and software of the application processor 18 and/or the delivery processor 20 to take over their role to complete an infusion action when either of them or both are faulted.

Under a normal operational condition, the infusion pump control system 14 utilizes the delivery processor 20, the application processor 18, the safety processor 22, and the user-interface processor 16 to deliver a safe infusion. Through the communication bus 30, the application processor 18, after accepting a user's instruction by the user-interface processor 16, directs the delivery processor 20 to run the driving components such as a motor. The user-interface processor 16 displays the pump states and infusion setting data affecting infusion progress.

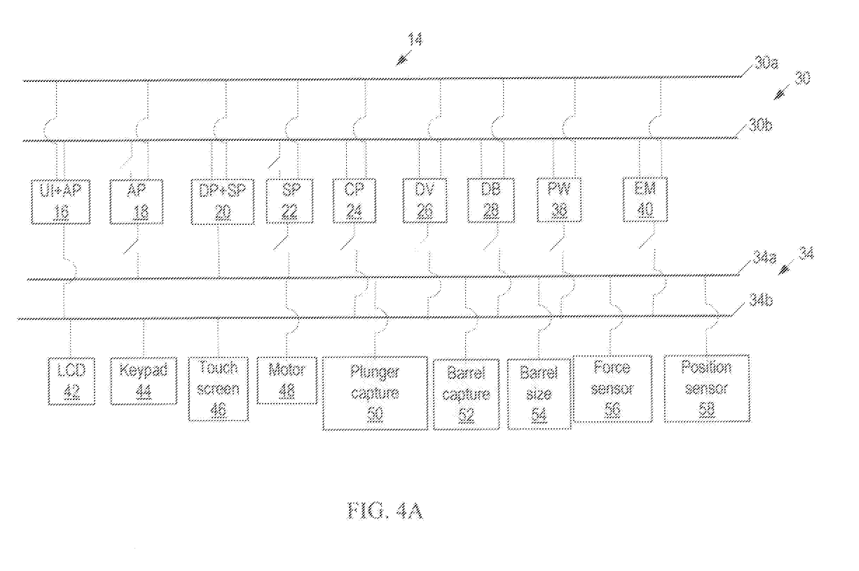

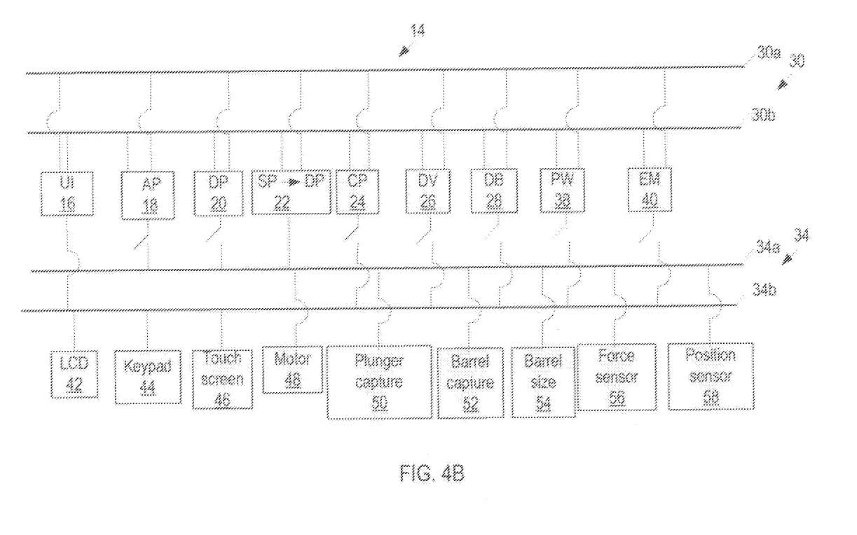

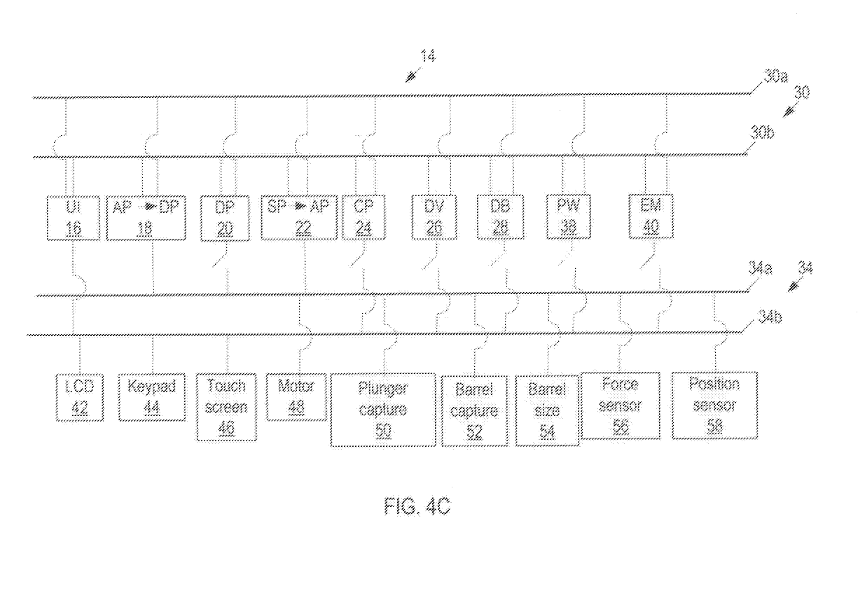

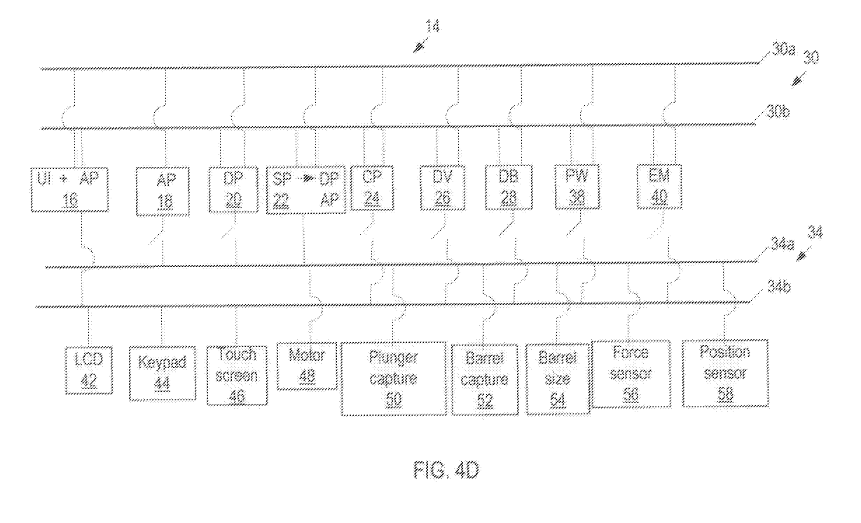

Referring to FIG. 4A, in a minimalistic scenario to sustain/complete a running infusion when both the application processor 18 and the safety processor 22 are faulted, the delivery processor 20 takes on the additional role of safety processor 2, and the user-interface processor 16 takes on the additional role of application processor 18, and the delivery processor 20 directly couples to the user-interface processor 16 to complete an active infusion. In the aforementioned situation, the communication bus 30, implementing a point-to-point communication between the delivery processor 20 and user-interface processors 16, supports automatic reconfiguration of the pump to permit the completion of an active infusion.

Referring to FIGS. 4B and 4C, when the delivery processor 20 fails, to permit active infusions to complete, in an embodiment, the safety processor 22 is capable of taking over the delivery processor 20, and in another embodiment, the application processor 18 is capable of taking over the delivery processor 20. In the aforementioned scenario, if the application processor 18 takes the role of the delivery processor 20, the safety processor 22 would take over the role of the application processor 18 as shown in FIG. 4C. When both the application processor 18 and delivery processor 20 become faulted, referring to FIG. 4D, either the safety processor 22 replaces both the delivery processor 20 and the application processor 18, or the safety processor 22 replaces the delivery processor 20 and the user-interface processor 16 take additional role of the application processor 18 to complete the infusion task. Referring to FIG. 4E, when only the application processor 18 fails, the safety processor 22 acts the role of the application processor 18 to complete an infusion processor.

In an embodiment, a processor of the plurality of processor, other than the user-interface processor 16, the application processor 18, the delivery processor 20, and the safety processor 22, that has the least sufficient circuit and software of the application processor 18, the delivery processor 20, and/or the safety processor 22 is able to replace the function of the application processor 18, the delivery processor 20 when such a processor also is connect to the sensor bus 34.

In an embodiment, a processor capable of replacing other processors constantly receives signals from such other processor indicating functional state of such other processors.

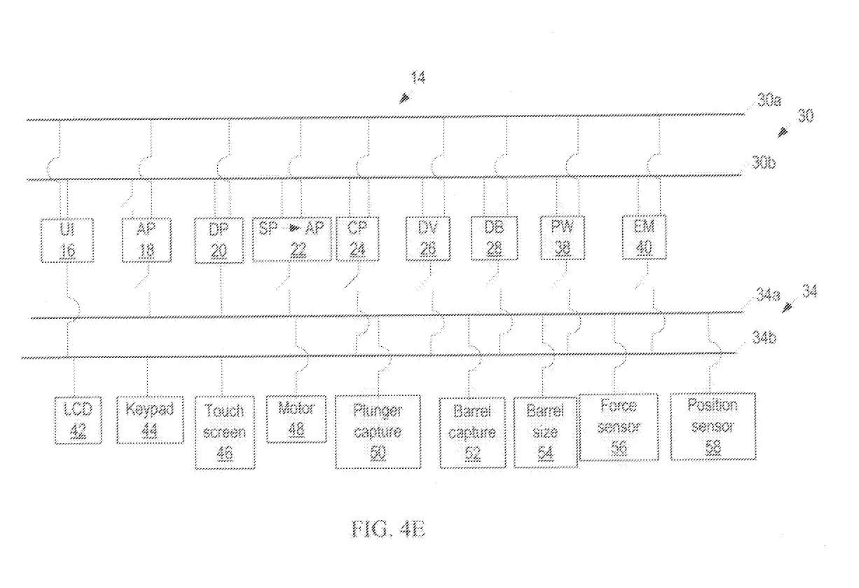

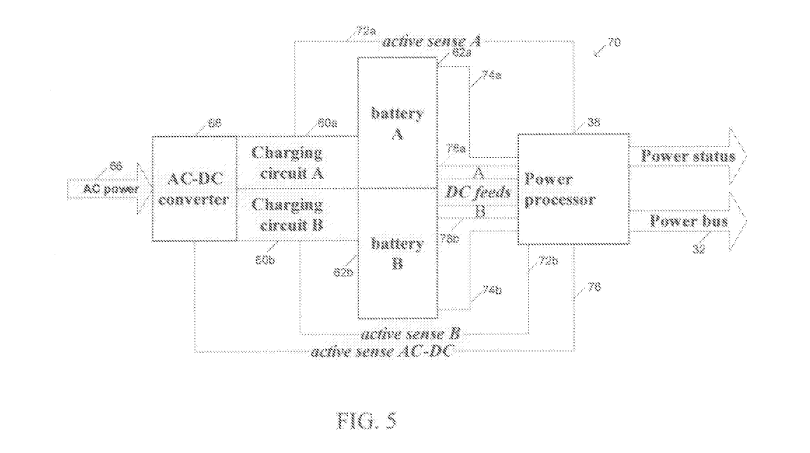

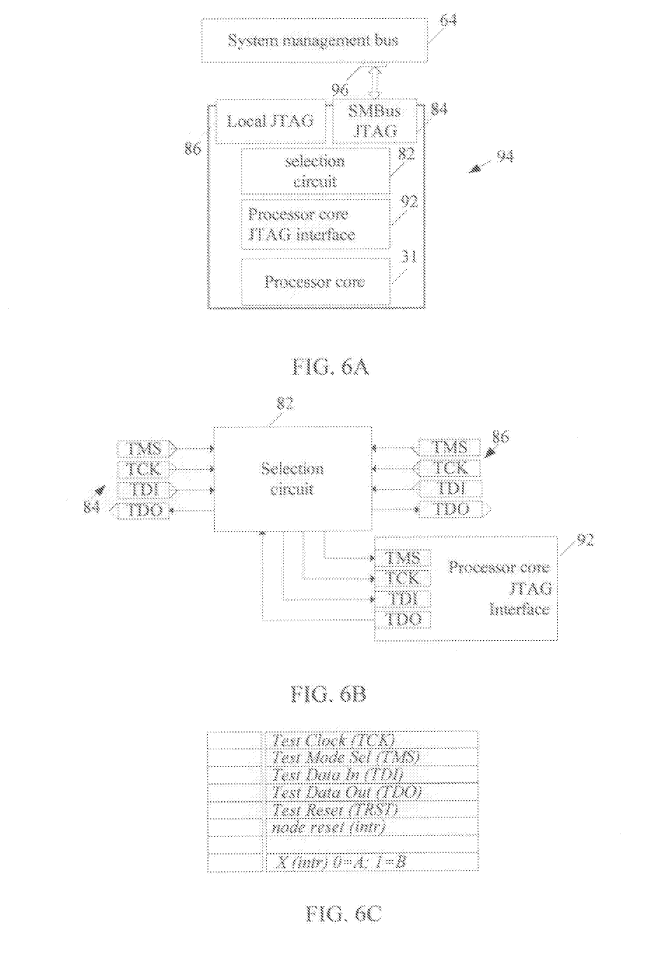

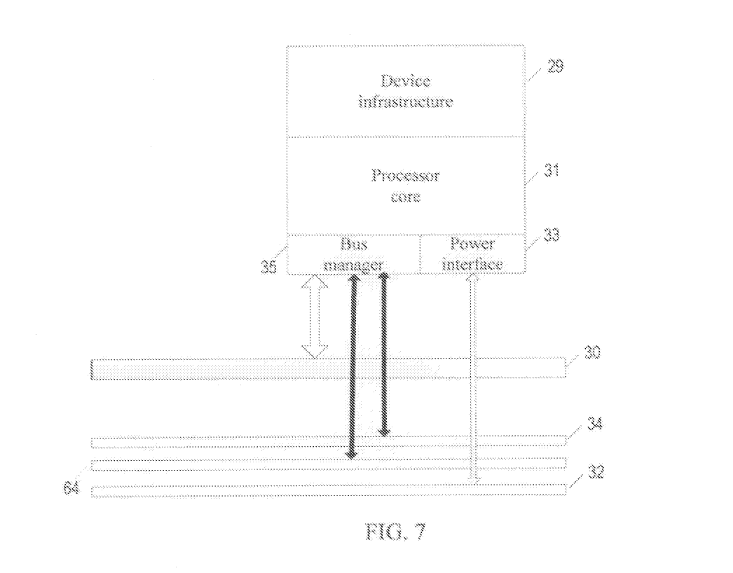

In an embodiment, referring to FIG. 7, the infusion pump control system 14 further comprises a system management bus 64, which interfaces with a power processor (PW) 38 to post power status to other processors in FIG. 5 and provides for a multi-dropped Joint Test Action Group (aka JTAG with TAP) 96 communicating with a multi-dropped JTAG selection mechanism 94 in FIGS. 6A and 6B.

The power processor 38 manages a power system 70 to switch among redundant power supplies over a power bus 32, and handles charging duties, power fault detection. The power system 70 is illustrated in FIG. 5. The power system 70 includes redundant, identical battery packs 62a and 62b and charging circuits 60a and 60b, insuring that clean power is delivered to the device circuitry in a highly-available manner. The infusion pump control system 14 draws power from a common power bus 32, which is connected to the power processor 38, supplying separate power planes to support the plurality of processors, the device infrastructure, and the motor(s).

The power processor 38 handles several failure conditions: loss/lack of AC power, loss of one or both charging circuits, absences of one or both batteries, failure of one or both batteries. Additionally, the power processor 38 performs several tasks, providing conditioned DC voltage to the power bus 32, monitoring the presence/state of the redundant batteries 62a and 62b, monitoring the state of the redundant charging circuits 60a and 60b, monitoring the availability of AC power 66, and managing power utilization.

Under a normal operating scenario, both the batteries 62a and 62b are installed and are completely functional, and the charging circuits 60a and 60b actively maintaining the power levels respectively of the batteries 62a and 62b by AC power 66 in conjunction with AC-DC converter 68. When one of the batteries 60a and 60b fails, the power processor 38 configures the in-bound power from the other battery that remains functional. In contrast, when both batteries 60a and 60b fail, the AC power source is used directly to maintain power.

The batteries 62a and 62b by a switch mechanism 74a and 74b, charging circuits 60a and 60b by active sense 72a and 72b, and AC-DC converter 68 by active AC-DC sense 76 constantly send the power processor 38 digital signals indicating the status of the batteries 62a and 62b, charging circuits 60a and 60b, and AC-DC converter 68. The batteries 62a and 62b are connected with the power processor 38 by DC Feeds 78a and 78b.

As stated previously, another function of the system management bus 64 provides for the multi-dropped JTAG 96 connected to selection mechanism 94 positioned in the boards of the plurality of processors, which is demonstrated in FIGS 6A and 6B, to support the capabilities—a firmware provisioning to and selectively debugging the processor cores 31 in the plurality of processors.

The multi-dropped JTAG selection mechanism 94 includes a SMBus JTAG connector 84 used to connected the processor core, with which the multi-dropped JTAG selection mechanism 94 is associated, to the multi-dropped JTAG 96 positioned in the system management bus 64, a local JTAG connector 86 for connecting the processor core, with which the multi-dropped JTAG selection mechanism 94 is associated, to a JTAG cable, a selection circuit 82, and a processor core JTAG interface 92. The multi-dropped JTAG 96 allows selecting of only one of the several processor cores connected, wither programming the specific flash memory of that processor cores or supporting a debugging session between the selected processor cores and an integrated development environment (IDE). However, although this works fine for provisioning the firmware of the processor cores, under many of the operational debugging scenarios, two or more processor cores are involved at the same time. Thus, to provide for the debugging of two or more processor cores, it is necessary to provide an ability to remove a specific processor cores from the multi-dropped JTAG 96 without disrupting the multi-dropped JTAG 96's access to another processor core.

FIGS. 6A and 6B are an illustration how the multi-dropped JTAG selection mechanism 94 is used to support this capability. In the multi-dropped JTAG selection mechanism 94, a SMBus JTAG connector 84 is continually connected to the multi-dropped JTAG 96 while the local JTAG connector may or may not be connected to a JTAG cable. When a JTAG cable is not connected to the local JTAG connector 86, the selection circuit 82 routes the signal sent from SMBus JTAG connector 84 to the processor core 31 through the processor core JTAG interface 92. In contrast, when a JTAG cable is connected to the local JTAG connector 86, the selection circuit 82 removes the SMBus JTAG connector 84 from the multi-dropped JTAG 96 and routes the local JTAG connector 86's signals to the processor core JTAG interface 92.

As a simple example, consider a debugging session involving two processor cores. One of the pair can be directly addressed using the multi-dropped JTAG 96, while the other processor core is addressed by the use of the local JTAG connector 86. The processor core 31 that is addressed using the local JTAG connector 86 is not connected to the multi-dropped JTAG 96 as long as a JTAG cable is connected to the local JTAG connector 86; the SMBus JTAG 84 connector's signals are bypassed and continued to allow the other processor core communication with the multi-dropped JTAG 96.

FIG. 6C is a layout of the system management bus 64. The system management bus 64 contains the multi-dropped JTAG 96, and a processor reset (in-bound, interrupt).

Referring to FIG. 7, each of the plurality of processors has a different device infrastructure 29 but the same processor core 31 and the same power interface 33 and a partially same bus manager 35. The processor core 31 and the bus manager 35 and the power interface 33 of each module are designed to have identical circuits and to be controlled by identical software, making it possible to use the common communication bus 30 and the power bus 32. Depending on the specific functions, the bus manager of each of the plurality of the processors that need use the same sensor bus also have an identical circuit and to be controlled by the identical software to use the same sensor. For example, application processor 18, delivery processor 20, and safety processor 22 have an identical circuit and are controlled by the identical software to use the sensor bus 34.

The processor core 31 can be a microprocessor, microcontroller, or a digital signal processor, depending on the preference of a designer of this control system.

The infusion pump control system 14 further comprises a dataset/database processor (DB) 28 that manages all manner of storage requirements for drug & syringe libraries, firmware, pump transaction history and pump fault/maintenance histories. The dataset/database processor 28 is expandable as the central repository for all application specific data and provides redundant stores to handle potential failures in recording media.

The infusion pump control system 14 further comprise a communications processor (CP) 24. It provides connections to various communication networks related to pump activity. Three general networks are accommodated: device-level (inter-pump), local-area (LAN) and wide-area (WAN). Additionally, the communication processor 24 supports web-based monitoring to application-critical & management functions.

The infusion pump control system 14 further comprises a device processor (DV) 26 that provides access to various serial communication capabilities (RS232/422/485, USB, IRDA, RFID) to permit attachment to various external devices.

The infusion pump control system 14 further comprise at least one expansion modules(EM) 40 for future software expansion and a scalable design that permits the inclusion of processor modules for future expansion/upgrade.

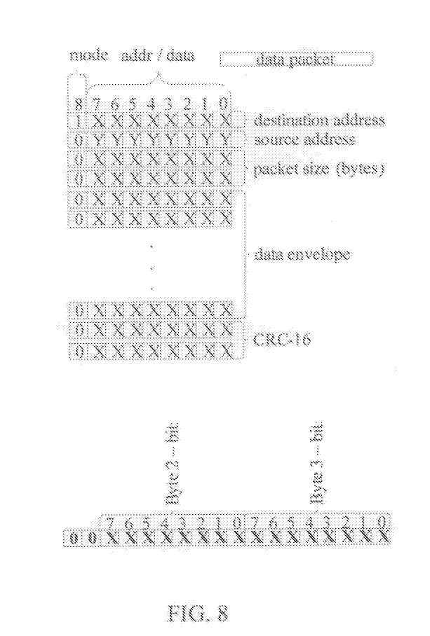

The device-level communication protocol for the communication bus 30 is shown in FIG. 8. The device-level communication protocol supports the transfer of simple, unformatted data packets with provisions for both point-to-point and broadcast message types over the communication bus 30.

Device-level data packets include following components: destination address, source address, packet size, data envelope, and cyclic redundancy check (CRC). The entire data packet is byte-oriented, where the most significant bit (MSb) of each byte is used to determine the start of a new packet.

The MSb of the byte containing a destination address is always set to one (1). The remaining bits in the destination address dictate whether the type of data packet being transmitted is a point-to-point data packet or a broadcast data packet. If bits 0:7 contain anything other than zero, a point-to-point data packet is defined and the value is the address of the destination processor. The processor assigned to the specific destination address accepts/processes the entire incoming packet, while the other processors continue to look for a byte with an MSb of one (1). If bits 0:7 contain a value of zero, a broadcast data packet is defined. In this case, all processors accept the entire incoming packet and selectively process its contents. The MSb of bytes of the remainder of the data packet are always set to zero (0).

Bits 0:7 of this byte contain the address assignment of the processor that transmitted the data packet.

The packet size is limited to 2̂14−1 bytes. This value is generated by appending bits 0:7 from Byte 2 and bits 0:7 from Byte 3 in the lower order bits of a 16-bit unsigned integer. The diagram to the left shows the form of this concatenation, data envelop (Bytes 4:N−2) and CRC (Bytes N−1:N). The data envelop is starts at Byte 4 and continues for the number of bytes formed by the packet size component. There is not implied format for the contents of the data envelop; its definition/layout/context is defined by the firmware in the processor receiving the data packet. The bytes that make up the CRC for a 16-bit unsigned integer that is the CRC calculation for bytes 0:N−2. Byte N−1 is the most-significant byte (MSB) and Byte N is the least-significant byte (LSB) for the 16-bit value.

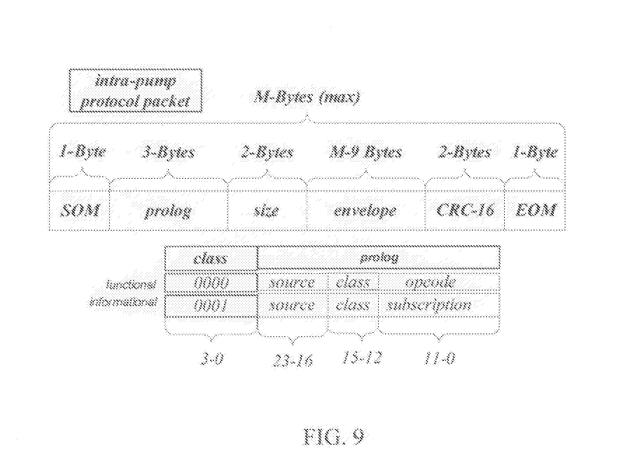

In FIG. 9, the application-level communication protocol supports the transfer of simple, unformatted data packets with provisions for both functional and information message types by the communication bus 30. There are two formats for an application packet: (1) intra-pump packets are intended for data exchange between processors, on the same pump, over the local data bus; and (2) inter-pump packets are intended for transmission between processors, not necessarily on the same pump, over a network medium (wired or wireless).

Each of these packets is carried as data in a device-level packet between processors, Intra-pump data packets (aka: local data packets) includes start-of-message byte (SOM), prolog (routing information), size (of the envelop), envelop (unstructured data content), CRC, and End-of-message byte (EOM).

The SOM and EOM bytes act as markers for the start and end of a data packet. They serve no other purpose.

The two bytes of the CRC form a 16-bit unsigned integer value that is calculated over the prolog, size and envelop components of the data packet. The SOM and EOM are not included in the CRC calculation.

The two bytes of the SIZE form a 16-bit unsigned integer value that contains the number of bytes held in the envelop component of the data packet.

The N-bytes of the ENVELOP contain a variable length, unstructured data vector (i.e. a counted byte string without a terminating marker). The context of the envelop contents is determined by the PROLOG component of the data packet.

The bytes containing the PROLOG component provide a structured context for the contents of ENVELOP. Consisting of a 3-byte sequence, the PROLOG component contains four distinct fields: (1) class; (2) source; (3) class (check copy); and op-code/subscription.

The class field is b 4-bit sequence that contains the type of data packet presented. The value of this field is repeated later in the packet as a 4-bit field, next to the source field, as a logical check. A value of “0000” indicates that the data packet is functional, meaning that it contains a command packet. A value of “0001” indicates that the data packet is informational, meaning that it contains tagged parametric data; sensor or calculated values that are tagged with a name and a data type. In both cases, the contents of the ENVELOP component contains data whose format is determined by the class value.

The source field is an 8-bit value that contains the ID of the processor that sent the application packet. It is not necessary to embed the ID of the destination processor as the data packet, when being analyzed, is already resident on the destination processor. The source ID provide so the destination processor can send a packet to the source processor as a response.

The final field is a 12-bit value that is determined by the class field value. If the data packet is a command packet (class=“0000”), the 12-bit field is an op-code field whose value holds the command being sent to the destination processor. In this case, the ENVELOP component contains untagged parametric values that are used to process the particular command represented by the op-code field value. If the data packet is an information packet (class=“0001”), the 12-bit field is a subscription field whose value hold the subscription-id being sent to the destination processor. In this case, the ENVELOP component contains tagged parametric values related to the specific subscription type.

The receipt of a functional packet requires that the destination processor return an acknowledgement to the source processor. This is done by raising the high-order bit of the opcode to “1”, placing the success and response parameters into an ENVELOP component and sending the response application packet to the processor that was the source of the in-bound application packet.

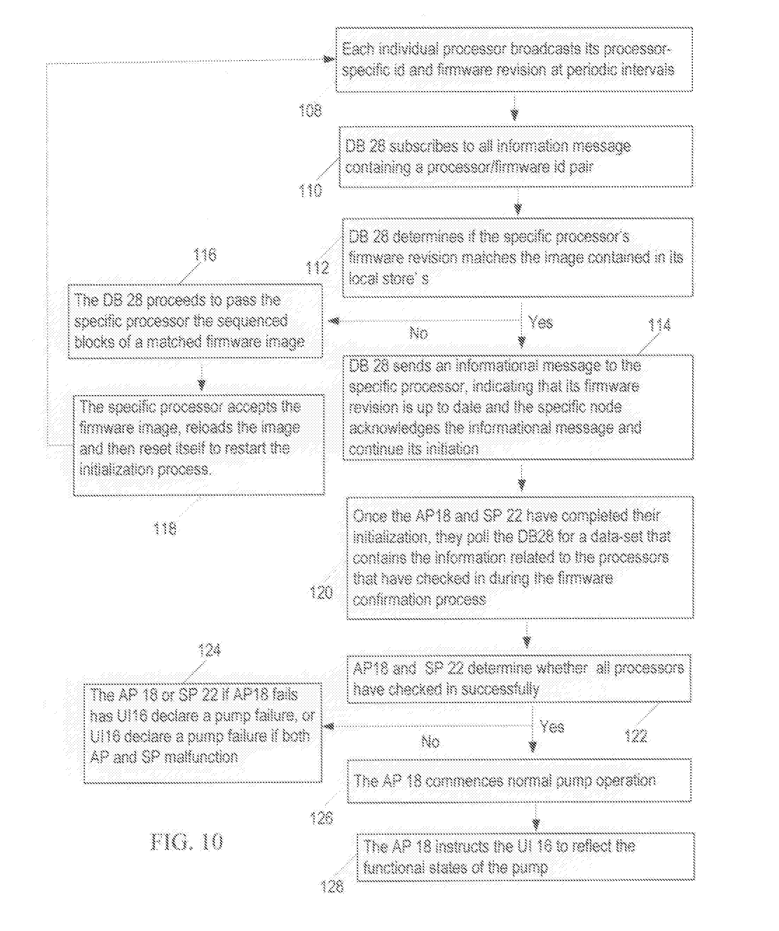

FIG. 10 shows a power-up initialization. In Step 108, each individual processor broadcasts its processor-specific id and firmware revision id on the system bus at periodic intervals. In Step 110, the database processor 28 subscribes to all information messages containing a processor/firmware id pair. For each processor/firmware id pair message received, the database processor 28 determines if the specific node's firmware revision matches the image contained in the local store in Step 112. If “yes,” in Step 114, the Database processor 28 sends an informational message to the specific processor, indicating that its firmware revision is up to date; the specific node acknowledges the informational message and continue its initialization profile. If “no”, the database processor 28 proceeds to pass the specific processor the sequenced blocks of its matched firmware image in Step 116, and in Step 118, the specific processor accepts the firmware image, reloads the image and then reset itself to restart the initialization process.

In Step 120, once the application processor 18 and safety processor 22 have completed their initialization, they poll the database processor 28 for a data-set that contains the information related to the processors that have checked in during the firmware confirmation process. This polling continues until all processors have checked in or N unsuccessful polls have been performed. Step 122 makes an inquiry as to whether all processors have check in successfully. If “yes,” the application processor 18 commences a normal pump operation in Step 124. If “no,” the application processor 18 (or the safety processor 22 if the application processor 18 has not responded) declares a pump failure, or the user-interface processor 16 declare a pump failure if both application processor 18 and safety processor 22 malfunction. Once the user-interface processor 16 has checked in, the application processor 18 instructs the user-interface devices 16 to reflect the functional state of the pump in Step 128. The infusion pump control system 14 allows a user to start to operate the infusion only after the infusion pump control system 14 has completed the initiation.

The process in FIG. 10 is a minimal scenario for firmware provisioning to utilize the contents of the local data store on the database processor 28. Updating to the firmware of a specific processor utilizes either the resources of the communications processor 24 or the device processor 26. A restart of the pump would be necessary and sufficient to complete the firmware update of the entire pump. The ability to update the firmware of a specific processor in the system means that firmware updates can be selectively provisioned.

It is assumed that the initial firmware provisioning for all processors in a pump is performed at the time of manufacture. The local data store is initialized with the firmware images loaded during manufacturing using a specific device header on the device processor 26 reserved for manufacturing. This initial load is performed using the multi-dropped JTAG 96. It is envisioned that the local data store is initialized. In an embodiment, a USB cable capability on the device processor 26 (for local data store access) is considered for this purpose.

The above examples are illustrative only. Variations obvious to those skilled in the art are a part of the invention. Additionally, the present invention does not require that all the advantageous features and all the advantages need be incorporated into every embodiment.

What is claimed is:

1. A infusion pump control system having at least one syringe, motor, at least one user-interface components, and at least one infusion pump-interface components, the infusion pump control system comprising a plurality of computing components discretely modularized to implement capabilities specific to infusion pump functions, and at least one communication bus, wherein the plurality of computing components communicate each other through the at least one communication bus.

2. The infusion pump control system of claim 1, wherein the plurality of computing components comprising processors.

3. The infusion pump control system of claim 1, wherein the plurality of computing components comprising
a. a user-interface processor,
b. a delivery processor,
c. an application processor accepting instruction from a user by the user-interface processor and directing the delivery processor to deliver an infusion, and
d. a safety processor independently acquires data, user-generated events and communications-related information from computing components other than the application processor and the user-interface processor to calculate pump state and infusion progress in order to monitor safety of the application processor.

4. The infusion pump control system of claim 3 further comprising at least one sensor bus, wherein the user-interface processor and the delivery processor continuously connect to the sensor bus, wherein the application processor and the safety processor connect to the sensor bus only in need.

5. The infusion pump control system of claim 4, wherein the application processor comprising at least sufficient software and hardware of the delivery processor and capable to take the roles of the delivery processor to complete an infusion task when the delivery processor fails.

6. The infusion pump control system of claim 4, wherein the application processor comprising the least sufficient software and hardware of the safety processor and capable of taking on the roles of safety processor when the safety processor fails.

7. The infusion pump control system of claim 4, wherein the delivery processor comprising at least sufficient software and hardware of the safety processor and capable of taking on the additional role of the safety processor when the safety processor fails.

8. The infusion pump control system of claim 4, wherein the safety processor comprising at least sufficient software and hardware of the application processor and capable of taking on the roles of the application processor to complete an infusion task when the application processor fails.

9. The infusion pump control system of claim 4, wherein the safety processor comprising at least sufficient software and hardware of the delivery processor and capable of taking on the role of the delivery processor to complete an infusion task when the delivery processor fails.

10. The infusion pump control system of claim 4, wherein the user-interface device processor comprising at least sufficient software and hardware of the application processor and capable of taking on the additional role of the application processor to complete an infusion task when the application processor fails.

11. The infusion pump control system of claim 2 further comprising at least one system manger bus.

12. The infusion pump control system of claim 11 further comprising a power bus, a power processor, and a power system having at least two power supplies, wherein when any of the at least two power supplies fails, the power processor automatically select remaining functional supplie(s) of the at least two power supplies, wherein the at least one system manager bus interfacing the power processor to post power status to other processors, wherein the power system provides power to the infusion pump control system by the power bus.

13. The infusion pump control system of 12, wherein the at least two power supplies including a plurality of DC power resources, and more than one charging circuits respectively charging the plurality of DC power resources coupling with an AC-DC converter.

14. The infusion pump control system of claim 11, wherein the system management bus providing for a multi-dropped JTAG connected to a multi-dropped JTAG selection mechanism, wherein the multi-dropped JTAG selection mechanism comprising a SMBus JTAG connector capable of connecting to the system manger bus, a local JTAG connector capable of connecting to a JTAG cable, a processor core JTAG interface, and a selection circuit to select to route a signal from either SMBus JTAG connector or the local JTAG connector to a processor core by the processor core JTAG interface with which the processor core JTAG interface is associated.

15. The infusion pump control system of claim 1, wherein the at least one communication bus use an arbitration mechanism.

16. The infusion pump control system of claim 4, wherein the at least sensor bus use an arbitration mechanism.

17. The infusion pump control system of claim 1, wherein the plurality of computing components comprising an expandable dataset/database processor.

18. The infusion pump control system of claim 3, wherein the application processor comprising a finite state machine to defines the state of the pump operation at any time point.

19. The infusion pump control system of claim 4, wherein the sensor bus is connected to at least one user-interface component and at least one infusion pump-interface component, wherein the sensor bus has a singular set of signal paths for each of the at least one user-interface component and at least one infusion pump-interface component.

20. The infusion pump control system of the claim 19, wherein at least one of the at least one user-interface component and the at least one infusion pump-interface components is redundant.

21. The infusion pump control system of claim 2, wherein each of the plurality of processor comprising a different device infrastructure but the identical processor core, and a sufficient identical bus manager for using a common bus.

22. The infusion pump control system of claim 1 comprising two communication buses, wherein both are connected to the plurality of computing component, but only one of them is in continuous use, wherein when the one in use malfunctions, the other one automatically communicate with the plurality of computing components.

23. The infusion pump control system of claim 1, wherein the plurality of computing components has sufficient redundancy to complete an infusion when a computing component of the plurality of computing components fails.

24. The infusion pump control system of claim 1, further comprising a device protocol and an application protocol with which the plurality of computing component communicate over the at least one communication bus.

25. The infusion pump control system of claim 1, wherein the plurality of the computing components comprising an expandable database processor managing all manner of storage requirements for drug & syringe libraries, firmware, pump transaction history and pump fault/maintenance histories

26. The infusion pump control system of claim 1, wherein the plurality of the computing components comprising a communication processor providing a connection to a network.

27. The infusion pump control system of claim 1, wherein the plurality of the computing components comprising a device processor that permits attachment to various external devices.

28. A method for operating an infusion pump, the method comprising
(1) Providing a plurality of computing components discretely modularized to implement capabilities specific to infusion pump functions.
(2) Providing at least one communication bus, wherein the plurality of computing components communicate each other through the at least one communication bus,
(3) Providing at least one sensor bus,
(4) Providing at least one syringe connecting to the at least one sensor bus where the plurality of computing components collects data of the syringe that contains a fluid, and
(5) Providing at least one pump, connecting to the at least one sensor bus where the plurality of computing components collects data of the pump, to pump the liquid directed by the plurality of computing components.

29. The method for operating an infusion pump of claim 28 further comprising providing a sufficient redundancy to at least one of the plurality of computing components to complete an infusion when said at least one of the plurality of computing components fails.
